(19) United States
(12) Patent Application Publication
(10) Pub. No.: US 2002/0159971 A1
(43) Pub. Date: Oct. 31, 2002

(54) Methods and compositions for preventing and treating neutrophil-mediated diseases

(76) Inventors: Michel Houde, Coteau-du-Lac (CA); Ghislain M. M. Opdenakker, Gent (BE); Rosemonde Mandeville, Sainte-Therese (CA)

Correspondence Address: MYERS BIGEL SIBLEY & SAJOVEC, PO BOX 37428, RALEIGH, NC, 27627, US

(21) Appl. No.: 10/082,148

(22) Filed: Feb. 25, 2002

Related U.S. Application Data
(60) Provisional application No. 60/275,550, filed on Feb. 23, 2001.

(51) Int. Cl. A61K 38/19; A61K 39/395

(52) U.S. Cl. 424/85.1; 424/146.1

(57) ABSTRACT

The present invention relates to methods and pharmaceutical compositions for the treatment of neutrophil-mediated diseases in humans and animals. More particularly, the present invention is concerned with methods and compositions for preventing and treating diseases such as cancer as well as acute and chronic inflammation by specifically neutralizing the biological activity of a neutrophil-secreted matrix metalloproteinase (MMP). According to a preferred embodiment of the invention, the neutrophil-secreted MMP that is targeted is MMP-9 (gelatinase B). According to the invention, the beneficial effects conferred by the specific neutralization of a neutrophil-secreted MMP, and more particularly MMP-9, are not counterbalanced by the detrimental effects of a broad and non-specific inhibition of other MMPs.

### RELATED APPLICATION

This application claims priority from U.S. Provisional Application No. 60/275,550, filed Feb. 23, 2001, the disclosure of which is incorporated by reference herein in its entirety.

### BACKGROUND OF THE INVENTION

a) Field of the invention

The present invention relates to methods and pharmaceutical compositions for the prevention and treatment of neutrophil-mediated diseases in humans and animals. More particularly, the present invention is concerned with methods and compositions for preventing and treating diseases such as acute and chronic inflammation by specifically neutralizing the biological activity of a neutrophil-secreted matrix metalloproteinase (MMP).

b) Description of the prior art

### Neutrophils and Inflammation

Inflammation is a reaction of a tissue and its microcirculation to a pathogenic insult. It is characterized by the generation of inflammatory mediators and movement of fluid and leukocytes from the blood into the extravascular spaces.

Inflammatory diseases certainly represent a major threat to human health. In particular, pathologies arising from acute inflammation such as septic shock and ARDS (Acute Respiratory Distress Syndrome), are conditions for which no treatment is currently available. The mortality rate associated with these conditions is often over 50%. In the case of chronic inflammations, severe side effects are associated with some medications, particularly glucocorticoids.

Inflammation is often considered in terms of acute inflammation that includes all the events of the acute vascular and acute cellular response, and chronic inflammation that includes the events during the chronic cellular response and resolution or scarring. Generally speaking, acute inflammation is mainly mediated by neutrophils, whereas chronic inflammation is associated with the additional presence of macrophages and lymphocytes.

Neutrophils, which are also known as polymorphonuclear leukocytes (PMN), comprise 40 to 75% of the total circulating leukocytes, numbering 2500 to 7500 cells per cubic millimeter. They are the principal cells of acute inflammation and actively phagocytize invading microorganisms. Neutrophils comprise various types of granules that play a central role in neutrophil function (Slavkovsky, 1995), some specific granules containing gelatinase B (Cowland and Borregaard, 1999).

Many studies have demonstrated a major role of neutrophils in septic shock (Adams et al., 2001; Frey et al., 2000; Mariano et al., 2001; Sullivan et al., 1995), acute respiratory distress syndrome (ARDS; Adams et al., 2001; Geerts et al., 2001; Dunican et al., 2000), bacterial meningitis (Kieseier et al., 1999; Koedel and Pfister, 1999; Leppert et al., 2000), acute pancreatitis (Kuijpers et al., 1999), multiple organ failure (MOF; Adams et al., 2001), post-ischemic reperfusion (Lindsey et al., 2001), acute cellulitis (Sachs, 1991), abdominal aortic aneurysm (Spark and Scott, 2001), asthma (Sampson, 2000; Greener, 2000; Greener, 1999), osteomyelitis (Nurre et al., 1999; Chadha et al., 1999), Crohn's disease (Matsukawa et al., 1999), cystic fibrosis (Eichler et al., 1999), emphysema (Betsuyaku et al., 1999; Finkelstein et al., 1995), septic or bacterial pyelonephritis (Kooman et a., 2000; Roberts, 1993), rheumatoid arthritis (Matsukawa et al., 1999), septic arthritis (Marchevsky and Read, 1999; Rotbart and Glode, 1985), uveitis (Smith et al., 1998; Chan and Li, 1998), periodontitis (Leino and Hurttia, 1999), psoriasis (Terui et al., 2000), severe burns (Kuijpers et al., 1999), skin ulceration (San Mateo et al., 1999; Wilson et al., 1999), acute lung injury (Abraham et al., 2000; Dunican et al., 2000), pneumonia (Abul et al., 2001), trauma (Fisher et al., 2001; Kuijpers et al., 1999), severe early graft dysfunction (Fisher et al., 2001), brochioeactasis (Prikk et al., 2001; Sepper et al., 1995), chronic obstructive pulmonary disease (COPD; Betsuyaku et al., 2000), complications with hemodialysis (Kuijpers et al., 1999), hypersensitivity pneumonitis (Pardo et al., 2000), lung fibrosis (Pardo et al., 2000), herpes stromal keratitis (Thomas et al., 1997), restenosis (Welt et al., 2000), acute dermatitis (Mizgerd et al., 1997), glomerulonephritis (Zachem et al., 1997) and multiple sclerosis (Ziaber et al., 1995).

Some studies also suggest that neutrophils are also involved in the development of cancer, as these cells produce reactive oxygen species (ROS) that are known to regulate many genes and to induce DNA damage (Ernst, 1999). Alternatively, during invasion and metastasis, cancer cells may use to their advantage proteases such as MMP-9 produced by the neutrophils surrounding the tumor (Nielson et al., 1996)

Although the contribution of neutrophils to numerous inflammatory diseases is well documented, there is no suggestion nor demonstration in the prior art that neutrophil-secreted matrix metalloproteinases (MMPs) should specifically be blocked in order to provide a beneficial outcome of these diseases.

### Matrix Metalloproteinases (MMPs)

Recent studies suggested a major role played by matrix metalloproteinases (MMPs) in acute and chronic inflammations. In the case of acute inflammations, results have been reported for septic shock and acute respiratory distress syndrome (ARDS) (Carney et al., 1999; Delclaux et al., 1997; Gibbs et al., 1999; Nakamura et al., 1998; Ricou et al., 1996; Torii et al., 1997). In the case of chronic inflammations, the contribution of MMPs has been well established for conditions such as rheumatoid arthritis, multiple sclerosis and asthma (Cawston, 1998; Cuzner and Opdenakker, 1999; Holgate et al., 1999). However, conflicting results have also been reported (Betsuyaku et al., 1999; Ricou et al., 1996), leading to the hypothesis that MMPs are in fact a two-edged sword: they could potentially have detrimental effects, for example by degrading the extracellular matrix and destroying the architecture of some vital tissues. However, MMPs can also be beneficial for the remodeling of the tissues.

As mentioned hereinbefore, neutrophils contain gelatinase B, in the so-called gelatinase granules. Gelatinase B (also named MMP-9; type IV collagenase; 92 kDa gelatinase; EC 3.4.24.35) is a member of the MMP family and is released from various cell types, such as neutrophils, macrophages and lymphocytes. Except for neutrophils, all cells produce gelatinase B upon transcriptional stimulation, most of the times concomitantly with other MMPs, such as MMP1, MMP3 and MMP12, and also with natural inhibitors of MMPs such as TIMP-1. However, in the case of neutrophils, intracellular granules containing gelatinase B are present in the resting neutrophil; upon stimulation these cells quickly release gelatinase B from granules. In addition, neutrophils are the only cells to secrete a 120-130 kDa complex called NGAL in which gelatinase B is bound to lipocalin. Accordingly, and with the exception of MMP8, neither gelatinase A (MMP2) nor any other MMP is produced by neutrophils. Moreover, no TIMP is produced by neutrophils.

The cDNA sequence of human gelatinase B (MMP-9) has been published by Wilhelm et al. in 1989. Thereafter, a number of laboratories have developed MMP9 knock-out mice to study its role in various diseases, and more particularly in contact hypersensitivity (CHS; Wang M. et al. 1999), cerebral ischemia (Asahi et al., 2000; Wang et al., 2000), experimental autoimmune encephalomyelitis (EAE; Dubois et al., 1999), experimental bullous pemphigoid (BP, Liu et al., 1998), cardiac rupture (Heymans et al., 1999) and acute inflammation (Betsuyaku et al., 1999). Based on results of these studies, one could not assume that neutralization of MMP-9 biological activity would be a feasible or a suitable method for the treatment of inflammatory diseases since some authors suggest that MMP-9 plays no role in these conditions (Betsuyaku et al., 1999) and/or that its genetic depletion may be deleterious (Wang et al., 1999).

### MMP Inhibitors (MMPIs)

With regards to inflammatory diseases, several reports have shown a beneficial effect of MMP inhibitors (MMPIs) (Conway et al., 1995; Rasmussen and McCann, 1997; Wojtowicz-Praga et al., 1998; Yip et al., 1999). To the same extent, a few patents and patent applications teach that MMP inhibitors (MMPIs) may be used as a treatment for septic shock and other acute inflammations (WO 98/16506, U.S. Pat. No. 5,929,097, WO 93/23075, WO 98/03516). However, no conclusive results have been obtained so far concerning the beneficial effects of these MMPIs. A highly probable explanation for the lack of conclusive beneficial results is that all the MMPIs tested have a broad spectrum of inhibitory action, the MMPIs inhibiting both the beneficial and the detrimental MMPs. Accordingly, the beneficial effect conferred by the inhibition of one MMP is counterbalanced by the detrimental effect of the inhibition of one or several other MMPS.

For instance, most of the MMPIs tested so far are small chemical entities (SCE) targeting the active site of the MMPs. As this active site is similar for all MMPs (over 20 now), it means that all these MMPIs have a broad spectrum of inhibitory potential. For example, nanomolar concentrations of MARIMASTAT™ can inhibit the activity of MMP1, MMP2, MMP3, MMP8, MMP-9, MMP12 and other MMPs that have not been tested yet (Rasmussen and McCann, 1997). As a matter of fact, MMPIs directed against the active site of MMPs have even inhibitory potential against other metalloenzymes, such as carbonic anhydrase (Scozzafava and Supuran, 2000).

This broad range of inhibitory activities means that current MMPIs used for therapy could block the activity of the detrimental MMP(s) but could also block the activity of beneficial MMPs (and other metalloenzymes), which are required for the normal maintenance of human body functions and for tissue regeneration. In addition, some MMPs may have opposite effects: in that case, the net effect of the MMPI would be undetectable, the blocking of the activity of the pertinent MMP being masked by that of the irrelevant MMP. For instance, numerous authors have shown that MMP2 (gelatinase A) and MMP-9 (gelatinase B) have opposite effects (Maymon et al., 2000; Leppert et al., 2000; Neely et al., 2000; Takei et al., 1999; Mashuhara et al., 2000; Lijnen et al., 1998 and Bergers et al., 2000).

An antibody called REGA-3G12 is known to react specifically with gelatinase B (see EP 0 733 369; Paemen et al., 1995; Zhou et al., 1997; Pruijt et al., 1999). However, it has never been shown or suggested that this antibody could be used for specifically neutralizing the biological activity of a neutrophil-secreted MMP for the treatment of neutrophil-mediated diseases.

In view of the above, it is clear that there is a need for methods and compositions for the treatment of neutrophil-mediated diseases. More importantly, there is a need for more efficient methods and pharmaceutical compositions wherein neutrophil-secreted MMPs are specifically neutralized or blocked in conditions in which neutrophils appear to play a major role, as opposed to the simultaneous inhibition of other MMPs. There is more particularly a need for methods for prophylaxis and therapy wherein it is solely the biological activity of MMP-9 that is neutralized.

The present invention fulfils these needs and also other needs that will be apparent to those skilled in the art upon reading the following specification.

### SUMMARY OF THE INVENTION

An object of the invention is to provide methods and pharmaceutical compositions for the prevention and treatment of neutrophil-mediated disorders in humans and animals.

It is more particularly an object of this invention to provide methods and pharmaceutical compositions wherein a neutrophil-secreted matrix metalloproteinase (MMP) is specifically neutralized in conditions in which neutrophil-secreted MMPs appear to play a role, as opposed to the simultaneous inhibition of other MMPs.

A further object of this invention is to provide methods and compositions for the prevention and treatment of acute and chronic inflammation.

A further object of this invention is to provide methods and compositions for the prevention and treatment of cancers.

Another object of this invention is to provide a method for the prevention and treatment of a neutrophil-mediated inflammatory disorder, the method comprising the step of specifically neutralizing the biological activity of a neutrophil-secreted matrix metalloproteinase (MMP).

A further object of this invention is to provide a method for the prevention or treatment of neutrophil-mediated diseases in humans or animals, the method comprising administering to the human or animal a pharmaceutically effective amount of an inhibitor that neutralizes specifically the biological activity of a neutrophil-secreted matrix metalloproteinase (MMP).

The present invention also relates to the use of an inhibitor that neutralizes specifically the biological activity of a neutrophil-secreted matrix metalloproteinase (MMP) for the preparation of a pharmaceutical composition for the treatment and/or the prevention of a neutrophil-mediated disease in humans or animals.

The present invention further relates to the use of an inhibitor that neutralizes specifically the biological activity of a neutrophil-secreted matrix metalloproteinase (MMP), for the treatment and/or the prevention of a neutrophil-mediated disease in a human or an animal.

Furthermore, the invention provides a pharmaceutical composition for the treatment or prevention of a neutrophil-mediated disease in humans or animals, the composition comprising a pharmaceutically effective amount of an inhibitor that neutralizes specifically the biological activity of a neutrophil-secreted matrix metalloproteinase (MMP) and a pharmaceutically acceptable carrier or excipient.

According to a preferred embodiment of the invention, these objects are achieved by specifically neutralizing a single neutrophil-secreted MMP. More preferably the neutrophil-secreted MMP that is targeted is MMP-9 (gelatinase B). In a preferred embodiment, the gelatinase B inhibitor is an anti-gelatinase B antibody, and more preferably the neutralizing monoclonal antibody REGA-3G12.

A non-exhaustive list of pathological conditions that could be treated using the above-mentioned methods and/or the pharmaceutical compositions includes: septic shock, acute respiratory distress syndrome (ARDS), bacterial meningitis, acute pancreatitis, multiple organ failure (MOF), post-ischemic reperfusion, acute cellulitis, abdominal aortic aneurysm, asthma, osteomyelitis, Crohn's disease, cystic fibrosis, emphysema, septic or bacterial pyelonephritis, rheumatoid arthritis, septic arthritis, uveitis, periodontitis, psoriasis, severe burns, skin ulceration, acute lung injury, pneumonia, trauma, severe early graft dysfunction, brochioeactasis, chronic obstructive pulmonary disease (COPD), complications with hemodialysis, hypersensitivity pneumonitis, lung fibrosis, herpes stromal keratitis, vascular restenosis, glomerulonephritis, hypersensitivity, cardiac rupture arising as a complication of myocardial infarction, stroke and cerebral ischemia, traumatic brain injury and multiple sclerosis.

An advantage of the present invention is that it provides more effective means for the prevention and treatment of neutrophil-mediated diseases, and more particularly for the prevention and treatment of acute and chronic inflammation, as well as cancer. The invention allows the specific neutralization of the biological activity of a single neutrophil-secreted MMP without inhibiting the biological activity of one or several other beneficial MMPs. Therefore, the beneficial effects conferred by the specific neutralization of a neutrophil-secreted MMP, and more particularly MMP-9, are not counterbalanced by the detrimental effects of a broad and non-specific inhibition of many MMPs. This is an advantage of major medical importance since it not only improves the efficiency of the medical treatment, but it also reduces associated side effects.

Other objects and advantages of the present invention will be apparent upon reading the following non-restrictive description of several preferred embodiments, made with reference to the accompanying drawings.

### BRIEF DESCRIPTION OF THE DRAWINGS

FIGS. 1 and 2 depict the increased survival of newborn and adult MMP-9 knockout mice following an induced septic shock (injection of LPS) as compared to wild-type. MMP-9-null mice (homozygous −/−; C57BL/6 background) and their wild-type littermates (MMP-9 +/+; C57BL/6) were challenged intravenously (I.V.) with doses of lipopolysaccharide (LPS) (from Escherichia coli; Sigma) ranging from 50 to 600 μg to induce an endotoxic shock (50 mice per dose of LPS). Percentage of survival was evaluated on a daily basis. FIGS. 1 and 2 show the results obtained with young mice (4 weeks) and adult mice (>8 weeks), respectively.

FIG. 3 is a picture of a Western blot assay showing the specific binding to gelatinase B (MMP-9) of 3G12scFv, a recombinant derivative of the monoclonal antibody REGA-3G12.

FIG. 4 is a bar graph that shows the neutralizing effect of 3G12scFv on the biological activity of gelatinase B (MMP-9) purified from human neutrophil and absence of inhibition of gelatinase A (MMP2). The latter enzyme is the closest relative of gelatinase B within the MMP family.

### DETAILED DESCRIPTION OF THE INVENTION

A) Problematic

As mentioned previously in the “Background of the invention” section, cells involved in the inflammatory process, particularly neutrophils and macrophages, are known to secrete a considerable panel of proteases. Elevated levels of almost all of these proteases have been observed in acute as well as in chronic inflammatory conditions. Some of these proteases are involved in the initiation of the inflammation process while others are rather involved in the amplification or even in the resolution of the inflammation process. This situation still remains true in the case where the range of proteases considered is restricted only to the MMP family, as MMPs are involved in the degradation as well as in the remodeling of tissues. The control of the inflammatory response is the result of a delicate balance between various proteases, cytokines, chemokines and growth factors. Massive inhibition of whole families of proteases, such as it would be the case for inhibition of MMPs by tetracycline analogs or by hydroxamic acid-based inhibitors, could induce an imbalance at too many points along the cascade of events leading to inflammation or fibrosis: instead of being beneficial., this situation may actually lead to the aggravation of the final pathology and the induction of serious side-effects.

With regards to these considerations, the present inventors have thus taken the approach of blocking neutrophil-mediated diseases and more particularly neutrophil-mediated inflammatory disorders by specifically neutralizing the biological activity of a neutrophil-secreted matrix metalloproteinase (MMP), preferably a single neutrophil-secreted MMP, and more preferably gelatinase B (also named MMP-9; type IV collagenase; 92 kDa gelatinase; EC 3.4.24.35).

As used herein, “neutralizing” or “neutralization” means inhibiting, blocking, inactivating, affecting negatively and/or down-regulating, totally or at least partially, the biological activity of an enzyme (herein a neutrophil-secreted MMP).

“Specifically neutralizing” means neutralizing (see hereinabove) the biological activity of an enzyme (herein a neutrophil-secreted MMP) with a high level of specificity and without substantially inhibiting the biological activity of other protease(s) whose biological activity is considered beneficial (see hereinafter). Best specific inhibitors according to the present invention are those that exclusively neutralize the biological activity of a single selected neutrophil-secreted MMP without neutralizing the biological activity of other protease(s).

As used herein, “beneficial” refers to enzymes/proteins for which biological activity is desirable or advantageous, i.e. those enzymes/proteins that produce or promote a favorable result and/or are not harmful to human or animal health.

As mentioned previously, it is highly preferable according to the invention to neutralize specifically MMP-9 (gelatinase B) activity. Specific neutralization of MMP-9 is proposed to be more or equally efficient to broad inhibition of MMPs in pathological situations where neutrophils play a major role mainly because:

it will not affect other MMPs, such as MMP2 (gelatinase A), that may [later] play a constitutive and beneficial role in the resolution of the inflammatory process and in tissue remodeling;

the neutralization of gelatinase B will mainly affect neutrophil functions and not (or less) that of other cell types acting later in the inflammatory process, such as the macrophage. Macrophages will be much less affected because the production of gelatinase B by these cell types is almost always accompanied by the production of excess amounts of TIMP-1. Therefore, most of the gelatinase B released by the macrophages is almost immediately captured by its natural inhibitor;

gelatinase B is virtually absent from the circulation when the individual is “healthy”, in opposition to other MMPs such as MMP2 (gelatinase A);

neutrophils are the only cell type known to store gelatinase B intracellularly (gelatinase B granules): all the other cell types produce gelatinase B through a transcriptional/translational/secretory mode;

neutrophils produce only two MMPs: MMP-9 and MMP8;

neutrophils are the only cell type known to secrete gelatinase B without the concomitant secretion of TIMP-1, its natural inhibitor: this means that following neutrophil activation, the gelatinase B released from the granules is free to act on all the substrates available until TIMP is transcribed, translated, produced and secreted, these processes requiring several hours;

some of the substrates of gelatinase B which are activated and/or potentiated by gelatinase B such as IL-8, TFPI and IL-1, are well-known mediators of inflammation and coagulation responses.

B) Methods and Pharmaceutical Compositions of the Present Invention

The present application describes methods and pharmaceutical compositions for the prevention and treatment of neutrophil-mediated inflammatory disorders and neutrophil-mediated diseases in humans and animals.

Neutrophil-mediated inflammatory disorder includes all diseases in which an acute and/or chronic inflammation occurs and in which neutrophils are known to play a key role. Specific examples include septic shock, acute respiratory distress syndrome (ARDS), bacterial meningitis, acute pancreatitis, multiple organ failure (MOF), post-ischemic reperfusion, acute cellulitis, abdominal aortic aneurysm, asthma, osteomyelitis, Crohn's disease, cystic fibrosis, emphysema, septic or bacterial pyelonephritis, rheumatoid arthritis, septic arthritis, uveitis, periodontitis, psoriasis, severe burns, skin ulceration, acute lung injury, pneumonia, trauma, severe early graft dysfunction, brochioeactasis, chronic obstructive pulmonary disease (COPD), complications with hemodialysis, hypersensitivity pneumonitis, lung fibrosis, herpes stromal keratitis, restenosis, and glomerulonephritis.

Neutrophil-mediated diseases include all the neutrophil-mediated inflammatory disorders mentioned previously plus hypersensitivity, cardiac rupture arising as a complication of myocardial infarction, stroke and cerebral ischemia, and traumatic brain injury.

A number of methods for neutralizing the biological activity of an enzyme such as MMPs are well known. A first common approach consists of blocking the expression of the gene coding for the enzyme or blocking the translation of the RNA transcript(s) coding for the enzyme. Common well-known techniques and methods include targeted mutagenesis, transfer DNA (T-DNA) insertion mutagenesis, the use of ribozymes and of antisense oligonucleotides, to name a few. These methods could be used to reduce to practice the present invention.

Another approach for neutralizing the biological activity of an enzyme is to chemically block its function(s). This can be achieved using any suitable compound that interferes with the normal biological activity of the enzyme, without being toxic to the individual. Well-known suitable compounds include neutralizing antibodies directed against the enzyme, analogs and derivatives of neutralizing antibodies, peptides and proteins, chemical compounds and chemical conjugates, and any similar compounds or substances which interfere with the normal biological activity of the enzyme.

According to a first aspect, the methods of the invention comprise the step of specifically neutralizing the biological activity of a neutrophil-secreted matrix metalloproteinase (MMP).

In another aspect, the methods of the invention comprise the step of administering to a human or an animal in need thereof a pharmaceutically effective amount of an inhibitor that specifically neutralizes the biological activity of a neutrophil-secreted matrix metalloproteinase (MMP).

In a further aspect, the invention relates to the use of an inhibitor that neutralizes specifically the biological activity of a neutrophil-secreted matrix metalloproteinase (MMP) for the treatment or prevention of neutrophil-mediated diseases in a human or an animal or for preparing a pharmaceutical composition intended for such use. The invention further provides pharmaceutical compositions comprising such inhibitor(s) and a pharmaceutically acceptable carrier or excipient.

As mentioned previously, it is highly preferable according to the invention that the biological activity of a single neutrophil-secreted MMP be neutralized. Even more preferably, this neutrophil-secreted MMP is MMP-9 (gelatinase B).

According to a preferred embodiment of the invention, MMP-9 biological activity is neutralized with an anti-MMP-9 neutralizing antibody. As used herein, “antibody” and “antibodies” include all of the possibilities mentioned hereinafter: antibodies or fragments thereof obtained by purification, proteolytic treatment or by genetic engineering, artificial constructs comprising antibodies or fragments thereof and artificial constructs designed to mimic the binding of antibodies or fragments thereof. Such antibodies are discussed in Colcher et al. (1998). They include complete antibodies, F(ab′)₂ fragments, Fab fragments, Fv fragments, scFv fragments, other fragments, CDR peptides and mimetics. These can easily be obtained and prepared by those skilled in the art. For example, enzyme digestion can be used to obtain F(ab′)₂ and Fab fragments by subjecting an IgG molecule to pepsin or papain cleavage respectively. Recombinant antibodies are also covered by the present invention.

The antibodies may be humanized or chimerized. The CDRs may be derived from a rat or mouse monoclonal antibody. The framework of the variable domains, and the constant domains, of the altered antibody may be derived from a human antibody. Such a humanized antibody may sometimes be preferable since it elicits a negligible immune response when administered to a human as compared to the immune response mounted by a human against a rat or mouse antibody.

Alternatively, the neutralizing antibody may be an antibody derivative. Such an antibody may comprise an antigen-binding region linked or not to a non-immunoglobulin region. The antigen binding region is an antibody light chain variable domain and/or heavy chain variable domain. Typically, the antibody comprises both light and heavy chain variable domains, that can be inserted in constructs such as single chain Fv (scFv) fragments, disulfide-stabilized Fv (dsFv) fragments, multimeric scFv fragments, diabodies, minibodies or other related forms (Colcher et al. 1998). Such a derivatized antibody may sometimes be preferable since it is devoid of the Fc portion of the natural antibody that can bind to several effectors of the immune system and elicit an immune response when administered to a human or an animal. Indeed, such a derivatized antibody would not lead to immune complex disease and complement activation (type III hypersensitivity reaction)

Alternatively, a non-immunoglobulin region is fused to the antigen-binding region. The non-immunoglobulin region is typically a non-immunoglobulin moiety and may be an enzyme region, a region derived from a protein having known binding specificity, a region derived from a protein toxin or indeed from any protein expressed by a gene, or a chemical entity showing inhibitory or blocking activity(ies) against the targeted MMP. The two regions of that modified antibody may be connected via a cleavable or a permanent linker sequence. The antibody may be a human or animal immunoglobulin such as IgG1, IgG2, IgG3, IgG4, IgM, IgA, IgE or IgD carrying rat or mouse variable regions (chimeric) or CDRs (humanized or “animalized”). According to a preferred embodiment of the invention, the antibody is coupled to an anti-inflammatory cytokine, more preferably selected from the group consisting of IL-1 receptor antagonist, IL4, IL-6, IL-10, IL-11, IL-13, TGFβ and somatostatin. The antibody may also be conjugated to a carrier, such as serum albumin, in order to provide a specific delivery and prolonged retention of the antibody, either in a targeted local area or for a systemic application.

In a highly preferred embodiment, the present invention uses a monoclonal anti-MMP-9 antibody called REGA-3G12. This antibody is described in details in EP 0 733 369 which is incorporated herein by reference. The monoclonal antibody REGA-3G12 has been deposited at the Belgian Coordinated Collection of Microorganism (BCCM) on May 10^{th}, 1995 and was given accession number LMBP1366CB.

Even more preferably, the invention uses 3G12-scFv, a recombinant derivative of the monoclonal antibody REGA-3G12. The exemplification section of the present invention provides details on the production and specificity of the 3G12-scFv antibody. A person skilled in the art will understand that the invention is not restricted to this sole inhibitor and that other suitable specific neutrophil-secreted MMP inhibitors achieving the same or very similar functions could be used according to the present invention. For instance, one skilled in the art could produce, using well-known method, another anti-MMP-9 antibody. Such a person could also synthesize a synthetic peptide that could mimic the specific neutralization of REGA-3G12 to gelatinase B. Such peptide could be obtained after several rounds of panning of a phage display library in a system consisting of capture by a gelatinase B-coated matrix and subsequent elution with REGA-3G12. The peptide deduced from the sequence of the binding phage could be synthesized and used according to the methods and composition described herein. In addition, the smaller size of such synthetic peptide would, similarly to the 3G12-scFv, allow its passage through the damaged blood-brain barrier (BBB) so that it could eventually be used for the prevention and treatment of inflammatory CNS conditions such as bacterial meningitis or multiple sclerosis. A similar approach could probably be used for preparing other REGA-3G12 mimicking molecules such as nucleotides, peptide nucleic acids (PNA), non-peptidic molecules, haptamers or others. Therefore, the use of such molecules is considered to be within the scope of the present invention.

The specific neutrophil-secreted MMP inhibitor(s) and the pharmaceutical compositions comprising the same may be administered by any suitable route. For example, the gelatinase B inhibitor and the pharmaceutical composition may be given orally or nasally in the form of tablets, capsules, powder, syrups, etc., or by means of a spray, especially for treatment of inflammatory respiratory disorders such as ARDS and asthma. They may also be formulated as creams or ointments, especially for use in the treatment of skin disorders such as bacterial cellulitis, severe burns or leg ulcers. They may be formulated as drops, or the like, for administration to the eye and for use in the treatment of disorders such as uveitis. They may also be given parenterally, for example intravenously, intramuscularly, subcutaneously or intra thecally by injection or by infusion.

For preparing the specific neutrophil-secreted MMP inhibitor and the pharmaceutical compositions comprising the same, methods well-known in the art may be used. For example, oral administration may necessitate the use of a capsule coated with known coating agents to make sure that the inhibitor and the pharmaceutical compositions comprising the same are not digested or degraded in the intestinal tract. Any pharmaceutically acceptable carriers, diluents, excipients, or other additives usually used in the art, are suitable depending upon the desired method of administering it to humans or animals. For injectable solutions, excipients which may be used include but are not restricted to, for example, water, isotonic saline solution, isotonic glucose solution, polyols, glycerine, and emulsions or infusions for parenteral administration.

The pharmaceutical compositions of the invention may also contain preserving agents, solubilizing agents, stabilizing agents, wetting agents, emulsifiers, sweeteners, colorants, odorants, salts, buffers, coating agents or antioxidants.

The pharmaceutical compositions of the invention may also contain other therapeutically active agents such as inhibitors of other mediators of inflammation (e.g. anti-IL-1α, anti-IL-1β, anti-IL-2, anti IL-8, anti-IL-12, anti-TNFα, anti-IFNγ, and/or anti-LPS antibodies, inhibitors of elastase, anti-inflammatory cytokines such as IL-1 receptor antagonist, IL-4, IL-6, IL-10, IL-11, IL-13, TGFβ, somatostatin etc.). It may also be preferable in certain occasions to administer with the specific neutrophil-secreted MMP inhibitor, selective inhibitor(s) of another MMP. Anti-MMP antibodies, such as anti-MMP1, anti-MMP2 and anti-MMP8 antibodies, represent examples of specific MMP inhibitors. Examples of other inhibitors include: Ro-32-3555 (Roche, Basel, Switzerland) for MMP1, MMP8 and MMP13 and AG-3340 (Agouron Pharmaceuticals, San Diego, Calif., USA) for MMP2, MMP3, MMP-9 and MMP13.

The amount of specific neutrophil-secreted MMP inhibitor that is administered to a human or an animal or that is present in the pharmaceutical composition of the invention is a therapeutically effective amount. A therapeutically effective amount of inhibitor is that amount necessary for obtaining beneficial results without causing overly negative secondary effects in the host to which the inhibitor or composition is administered.

The exact amount of each inhibitor, of each of the components in the composition and amount of the composition to be administered will vary according to factors such as the type of the condition to be treated, the other ingredients in the composition, the mode of administration, the age and weight of the individual., etc. Without being bound by any particular dosage, it is believed that for instance for parenteral administration, a daily dosage of 0.1 to 100 mg/kg of REGA-3G12 neutralizing antibody (usually present as part of a pharmaceutical composition as indicated above) may be suitable for treating a typical adult. More suitably, the dose might be of 1 to 10 mg/kg. This dosage may be repeated as often as appropriate. Typically, administration may be 1 to 7 times a week. If side effects develop, the amount and/or frequency of the dosage can be reduced. A typical unit dose for the incorporation into a pharmaceutical composition would thus be at least 20 mg of REGA-3G12, suitably 20 to 1000 mg (for weights ranging from 40 to 100 kg).

C) Specific Non-restrictive Examples of Applications Using Anti-MMP-9:

(1) Treatment of Acute Inflammation

The invention also provides a method for treating a human with shock due to sepsis, comprising administering to this human a pharmaceutically effective amount of an anti-MMP-9 neutralizing antibody. According to a preferred embodiment, patients diagnosed with shock due to sepsis (with neutrophilia) within 12 hours after admission to the hospital are treated for 48-96 hours with the 3G12-scFv or the REGA-3G12 mAb. More preferably, the REGA-3G12 or the 3G12-scFv is provided as a sterile lyophilized preparation containing preservative agents, such as glycine or maltose. The REGA-3G12 monoclonal antibody or the 3G12scFv is next reconstituted with 10 ml sterile water and diluted to 100 ml with 5% aqueous dextrose solution. The REGA-3G12 monoclonal antibody or the 3G12scFv is then administered intravenously at doses ranging from 1 to 20 mg/kg/day (for example, a bolus injection of 50-1000 mg followed by a 96 h I.V. infusion of 1-20 mg/h using a volumetric infusion pump).

(2) Treatment of Chronic Inflammation by Topical Application

The invention also provides a method for treating ulcers chronic dermatologic inflammatory conditions such as acute dermatitis in a human, comprising applying on the skin of this human dressings impregnated or coated with an anti-MMP-9 antibody. According to a preferred embodiment, patients diagnosed with leg ulcers will receive dressings containing REGA-3G12 or 3G12-scFv at 24-48 h intervals for a is period of 2-8 weeks (or until complete healing of the ulcer, whatever occurs first). More preferably, the dressing contains a hydrocolloid matrix (e.g. gelatin, pectin, carboxymethylcellulose) to which REGA-3G12 or 3G12-scFv is combined. Alternatively, REGA-3G12 or 3G12-scFv can be mixed with a topical cream for the treatment of localized skin inflammations such as bacterial cellulitis.

(3) Treatment of Gastrointestinal Inflammation

The invention also provides a method for treating gastrointestinal inflammatory conditions in a human, comprising the oral administration to this human of a pharmaceutical composition, preferably a tablet, a capsule or a caplet, comprising as an active ingredient, an anti-MMP-9 antibody. According to a preferred embodiment, gastrointestinal inflammatory conditions such as Crohn's disease and ulcerative colitis are treated on a daily basis with REGA-3G12 or 3G12-scFv tablets. More preferably, REGA-3G12 or 3G12-scFv is encapsulated in soft gelatin tablets such as those manufactured by BANNER PHARMACAPS® (High Point, N.C., www.banpharm.com). These formulations allow the compound (REGA-3G12 or 3G12-scFv) to cross the gastrointestinal tract without being degraded and to reach the site of inflammation.

(4) Ex Vivo Gene Therapy With REGA-3G12 or 3G1 2-scFv

The invention also provides a method of ex vivo gene therapy of a human with an acute or a chronic inflammatory disease. The method comprises isolating from a human white blood cells (WBC; e.g. neutrophils, macrophages), transfecting at least a portion of the isolated WBC with a gene encoding for an MMP-9 specific inhibitor (e.g. an anti-MMP-9 antibody such as REGA-3G12 or 3G12-scFv); and re-injecting anti-MMP-9 expressing cells back to the human (see for instance IDM™, Paris, France; www.idmbiothech.com) so that the MMP-9 inhibitor be secreted concomitantly with MMP-9. The advantage of such method is that it ensures a specific and localized inhibition of the MMP-9 biological activity. A similar approach could also be used with an antisense molecule that would bind to the MMP-9 gene or RNA in order to directly block in situ the production of the MMP-9 enzyme.

(5) Ex Vivo Neutralization of Gelatinase B With REGA-3G12

The invention also provides an ex vivo method for neutralizing gelatinase B from a human or an animal. Such method could be particularly useful for the prevention or treatment of a neutrophil-mediated disease. The method comprises the step of filtrating the blood of a human or an animal diagnosed with an acute or a chronic inflammatory disease through an anti-MMP-9 matrix. Preferably, the matrix comprises an anti-MMP-9 antibody that specifically binds and neutralizes MMP-9. For instance, primary amino groups of the anti-MMP-9 antibody may be covalently attached to NHS (N-hydroxysuccinimide) group of the matrix or to a CNBr (Cyanogen Bromide)-activated matrix.

More preferably, the anti-MMP-9 antibody is the neutralizing monoclonal antibody REGA-3G12. In a preferred embodiment, the REGA-3G12 antibody is coupled to a resin, in a system similar to the PROSORBA™ column approved in US and Canada for the treatment of arthritis (see www.arthritisinsight.com/medical/meds/prosorba.html). A catheter is inserted in two different body sites of a patient. Blood is taken from one site and passed through a blood dialysis machine that separate the plasma from the blood cells. Gelatinase B binds with the REGA-3G12, removing it from the plasma. The plasma is then reunited with the blood cells and the blood is returned to the individual body via a second catheter at the second body site.

(6) Treatment of Cancers.

The invention further provides a method for treating cancers in humans. The method comprises administering to a human diagnosed with cancer a pharmaceutically effective amount of a specific MMP-9 inhibitor such as an anti-MMP-9 neutralizing antibody. Indeed, the present inventors expect that specific inhibition of MMP-9 would block or reduce the metastatic process generally associated with cancers and also block or reduce the inflammation associated with inflammatory cancers (e.g. breast, colon, lymphoma, pancreas, brain). It is also conceivable to use a gene therapy approach wherein a gene, encoding an anti-MMP-9 antibody such as REGA-3G12 or an antisense molecule that will bind to the MMP-9 gene or RNA, is inserted directly into the tumor cells.

As it will now be demonstrated by way of examples hereinafter, it is highly beneficial to neutralize the activity of a single neutrophil-secreted MMP (hereinafter MMP-9) as opposed to several MMPs. Indeed, the beneficial effect conferred by the neutralization of the single MMP is not counterbalanced by the detrimental effect of the inhibition of several MMPs.

### EXAMPLES

The following examples are illustrative of the wide range of applicability of the present invention and are not intended to limit its scope. Modifications and variations can be made therein without departing from the spirit and scope of the invention. Although any method and material similar or equivalent to those described herein can be used in the practice for testing of the present invention, the preferred methods and materials are described.

### Example 1

### Zymographic Analysis of Gelatinase B in Human Serum Samples

In a first set of experiments, serum and plasma specimens from three different donors (two septic shock patients and one normal donor) were tested for their gelatinase A and gelatinase B content by zymography (Masure et al., 1991). The relative activity of gelatinase A and gelatinase B was measured by densitometry and was expressed in arbitrary units (AU). The activity of the untreated serum from the healthy donor was considered as 100%.

Results presented in Table 1 hereinbelow show that the level of gelatinase B of the healthy donor (patient #1) increases by an 8-fold factor when the white blood cells of that donor are incubated with lipopolysaccharides (LPS), a well-known inflammatory agent from gram-negative bacteria. The gelatinase B levels of 2 patients having acute inflammation are also increased. In the case of patient #2, the increase of the gelatinase B level was correlated with the increase of the neutrophil counts. The gelatinase B level of patient #3 was very high at his arrival at the hospital. However, no significant increase of gelatinase A was observed neither between the three patients nor between the samples of the same patient collected at different times. These results demonstrate that two MMPs behave very differently in terms of induction in inflammatory situations, although these two MMPs, namely gelatinase A (MMP2) and gelatinase B (MMP-9) have been shown to cleave a very similar range of substrates. As the level of gelatinase A remains the same, one can think that this enzyme is required for the maintenance of the health of the individual and that its inhibition could have a detrimental effect. On the other hand, the levels of gelatinase B are absent or very low in the absence of inflammation and are increased by inflammatory stimuli. Consequently, the specific neutralization of gelatinase B may provide a beneficial effect to the patient, with non-significant or limited side effects, as gelatinase A is not affected by the therapy.

In addition, the detection or measurement of gelatinase B levels in biological fluids, such as serum, plasma, urine, cerebrospinal fluid (CSF), bronchoalveolar lavages (BALs) and others, may have a diagnostic utility as these levels give an indication of the activation of neutrophils in conditions where these cells are thought to play a significant role. The gelatinase B titer can then be expressed in terms of total content (proactive+active moieties), of active gelatinase content or of gelatinase index (content of gelatinase B/content of gelatinase A; either in active or proactive forms).

TABLE 1
Zymographic Analysis of Gelatinase B in Human Serum samples

| Identification | | Time | In vitro | [Gelatinase B] | | [Gelatinase A] | |
|---|---|---|---|---|---|---|---|
| Patient | Description | (days) | Treatment | Units | % | Units | % |
| #1 | Healthy Donor | T0 | Serum | 3,3 | 100% | 4,8 | 100% |
| | | | 6 h (Control) | 4,8 | 145% | 5,4 | 113% |
| | | | 6 h (LPS) | 26,5 | 803% | 6,7 | 140% |
| #2 | Woman with septic | T0 | | 5,7 | 173% | 4,7 | 98% |
| | shock (very low level of | T2 | | 5,3 | 161% | 4,8 | 100% |
| | monocytes; high levels | T3 | | 8,4 | 255% | 4,7 | 98% |
| | of neutrophils, | | | | | | |
| | particularly at day 3 | | | | | | |
| | (T3)) | | | | | | |
| #3 | Man with septic shock; | T0 | | 28,2 | 855% | 4,6 | 96% |
| | died at the day of arrival | | | | | | |
| | at the hospital | | | | | | |

### Example 2

### Specific Inhibition of MMP-9 Activity in Mice has a Beneficial Effect on Endotoxic Shock Survival

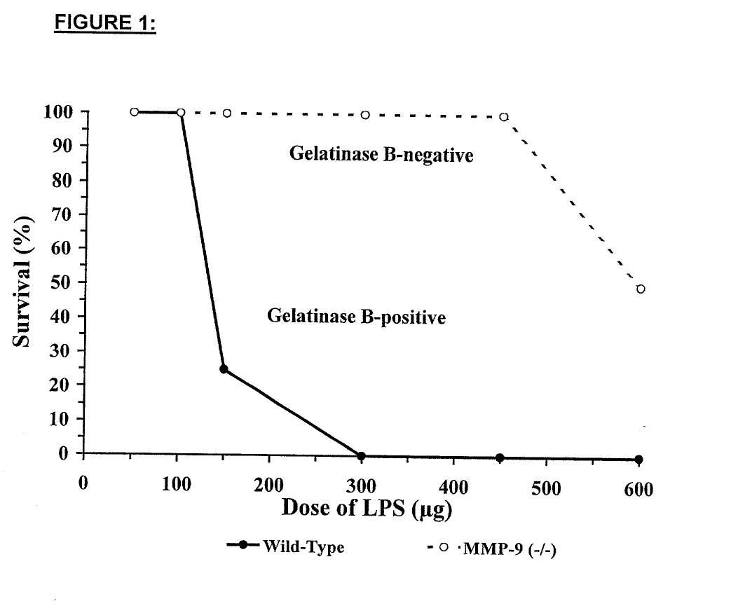

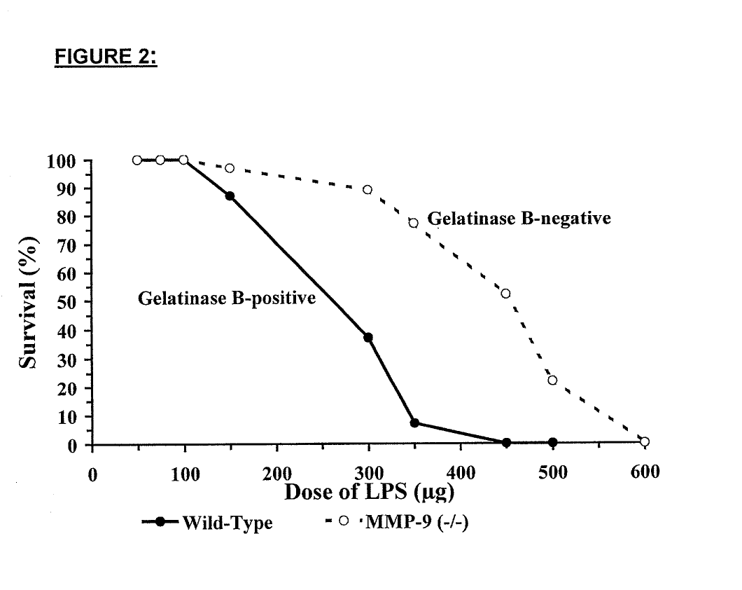

In a second set of experiments, MMP-9-null newborn mice (homozygous −/−) and their wild-type littermates (MMP-9 +/+) were challenged with lipopolysaccharide (LPS) to induce an endotoxic shock. The MMP-9-null mice are knocked out mice into which the MMP-9 gene has been deleted by the replacement of exons and corresponding introns 3-7 of the mouse gelatinase B gene by the neomycin resistance gene (Dubois et al., 1999). As shown in FIG. 1, the LD₅₀ (lethal dose inducing 50% mortality) of LPS was 5 to 10 times higher for MMP-9-null mice than for MMP-9-positive mice. These results demonstrate that specific inhibition of a single MMP activity such as the MMP-9 activity has a beneficial effect on endotoxic shock survival. In the case of adult mice, the LD₅₀ of LPS was 2 times higher for MMP-9-null mice than for MMP-9-positive mice (FIG. 2). These results also suggest that alternate mechanisms may have been used by the adult mice to partially compensate for the absence of gelatinase B.

### Example 3

### Use of an Anti-MMP-9 Antibody Derivative for Preventing and Treating Inflammatory Responses

i) Binding of 3G12-scFv

In a third set of experiments, a recombinant derivative of the monoclonal anti-MMP-9 antibody REGA-3G12 (3G12-scFv) was prepared and evaluated using Western Blot analysis. The 3G12-scFv that was used bears a histidine tag (His₆) at its C-terminal extremity, allowing the specific binding of an anti-His₆ monoclonal antibody (Qiagen, Germany). Briefly, 3G12-scFv was prepared by transforming Escherichia coli HMS174 (DE3) cells with a plasmid containing the T7 promoter and the cDNA coding for the 3G12-scFv fused to a histidine tag (His6). A bacterial clone was selected and grown in the appropriate medium. The expression of 3G12-scFv was next induced with IPTG. The 3G12-scFv protein was recovered from the bacterial pellet and purified by affinity chromatography using Ni-NTA agarose (Qiagen, Hilden, Germany).

Different preparations of human gelatinase A (MMP2) and B (MMP-9) were run in a SDS-PAGE and next transferred on a nylon membrane. Commercial preparations (Calbiochem) of monomer and dimer MMP-9 (gelatinase B), purified from human neutrophils, as well as MMP2 (gelatinase A) purified from human fibroblasts, were used. The blotted membrane was subsequently exposed to: (1) the 3G12-scFv (2 μg/ml); (2) an anti-His₆ mAb; and (3) an anti-mouse IgG mAb conjugated to horseradish peroxidase. The reaction was revealed using the SuperSignal Substrate™ (Pierce, USA).

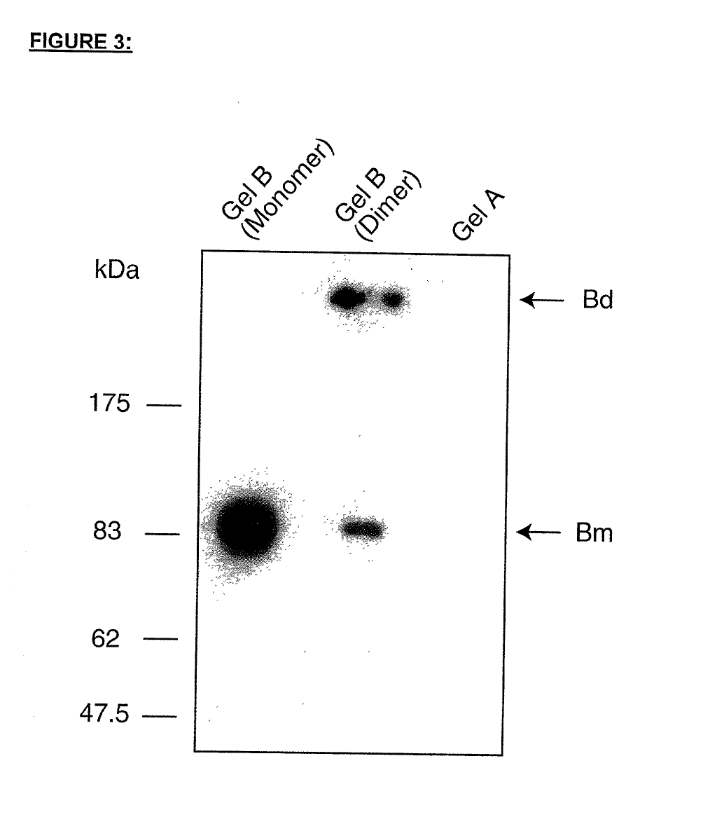

Results presented in FIG. 3 show that 3G12-scFv specifically bound to monomer and dimer gelatinase B (Gel B) purified from human neutrophils, as indicated by the two arrows. On the other hand, no band is visible in the lane containing the human fibroblast gelatinase A (Gel A).

ii) Neutralizing or Blocking Activity of 3G12-scFv

Next, the neutralizing or blocking activity of 3G12-scFv was evaluated using an assay based on the degradation of biotinylated gelatin (Bio-Gel™), a substrate of gelatinase A and B. The subsequent capture of non-degraded Bio-Gel™ at the surface of a streptavidin-coated microplate was measured by the spectrophotometric detection of Bio-Gel™ molecules labeled with a streptavidin-peroxidase conjugate.

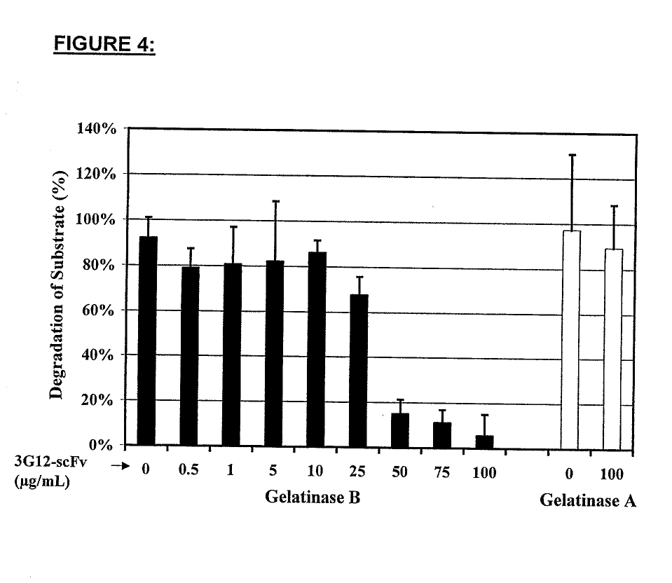

A dose-response titration of 3G12-scFv was also performed. Results illustrated in FIG. 4 demonstrate that doses of 3G12-scFv ranging from 25 to 100 μg/ml could significantly inhibit the degradation of Bio-Gel™ by 100 ng/ml of gelatinase B (p<0.05). The source of gelatinase B is a commercial preparation purified from human neutrophils (Calbiochem, Calif., USA). On the other hand, a dose of 100 μg/ml of 3G12-scFv had no effect on the degradation of Bio-Gel™ by 100 ng/ml of gelatinase A (commercial preparation of human fibroblast gelatinase A).

These results demonstrate that 3G12-scFv can specifically neutralize the activity of MMP-9 (gelatinase B) while having no effect on the activity of MMP2 (gelatinase A). Since the latter enzyme is the closest relative of gelatinase B within the MMP family, absence of neutralization of gelatinase A (MMP-2) by 3G12-scFv constitutes a proof of selectivity of 3G12-scFv in the neutralization of gelatinase B (MMP-9).

iii) Effect of 3G12-scFv on Inflammatory Responses

Based on the previous results showing that 3G12-scFv binds to gelatinase B and neutralizes its enzymatic activity in a specific manner, we next tested the effect of the REGA-3G12 fragment on a major biological process of inflammation, namely the migration of cells through an extracellular matrix. To evaluate this effect, a gelatin layer was deposited in the upper part of a Boyden chamber. A chemoattractant (enriched medium from U-87MG cells) was added in the lower chamber. Different cell types were added in the upper chamber, in the presence or absence of 3G12-scFv (50 μg/ml). After a 4 hours incubation at 37° C. (in the presence or absence of chemoattractant), the number of cells present in the lower chamber was counted. This number represented the number of cells having migrated through the gelatin layer.

The percentage of inhibition was determined using the following formula:

% Inhibition=1−[cells having migrated in presence of 3G12-scFv] cells having migrated in presence of a control antibody

Results presented in Table 2 hereinafter show that 3G12-scFv can significantly inhibit the migration of neutrophils, endothelial cells (BAE) and fibroblasts (COS-7), three different types of cells involved in the inflammatory process. This inhibition is dose-dependent, as shown by the results obtained by treating BAE and COS-7 with 10 and 50 μg/ml of 3G12-scFv.

TABLE 2
Effect of 3G12-scFv on the Migration of Cells.

| | | | | | Inhibition of Migration | |
|---|---|---|---|---|---|---|
| | | | | | by 3G12 (μg/mL) | |
| Cell Line | Origin | Description | MMP-2 | MMP-9 | 10 | 50 |
| Neutrophils | Human | Neutrophils | − | + | nd | 65% |
| BAE | Bovine | Aortic Endothelial | + | + | 56% | 75% |
| COS-7 | Monkey | Kidney fibroblast | + | + | 29% | 97% |

Results presented in Table 3 show that 3G12-scFv can also significantly inhibit the migration of cancer cells of different origin, using an experimental model similar to the one presented in Table 2. In addition, these results show that the inhibitory effect of 3G12-scFv is not correlated to the total concentration of MMP-9 in the reaction medium, as detected by zymography. The migration of cancer cells which produce undetectable or very low amounts of MMP-9, such as HepG2 and U-87 MG, is also inhibited by 3G12-scFv. This absence of correlation can be due to the presence of natural inhibitors of MMP-9 (e.g. TIMP-1) in the medium, to the level of activation of the MMP-9 enzyme (ratio of proenzyme vs. activated enzyme) or to the need of highly amounts of MMP-9 only in pericellular microenvironments. Neutrophils can therefore contribute to the migration of these tumor cells since these cells: (1) produce MMP-9 without any concomitant production of TIMP-1; (2) produce several other proteases and ROS that can activate MMP-9; and (3) can come in close contact with tumor cells and secrete high amounts of MMP-9 in pericellular microenvironments.

TABLE 3
Effect of 3G12-scFv on Cancerous Cell Lines.

| | | | | Inhibition of | |
|---|---|---|---|---|---|
| | | | | Migration by | |
| | | | | 3G12-scFv | |
| | | | | (μg/ml) | |
| Cell Line | Origin | MMP2 | MMP-9 | 10 | 50 |
| Panc-1 | Pancreatic Carcinoma | 2+ | 1+ | 59% | 79% |
| HepG2 | Hepatocellular Carcinoma | 1+ | − | 31% | N.D. |
| Caki | Kidney carcinoma | 1+ | 1+ | 11% | 67% |
| U-87 MG | Glioblastoma | 1+ | ± | 8% | 58% |

### References

Throughout this paper, reference is made to a number of articles of scientific literature that are listed below:

Abraham E et al. Am J Physiol-Lung Cell Molec Physiol 2000; 279: L1137-L1145.

Abul H et al. Molec Cell Biochem 2001; 217: 107-112.

Adams J M et al. J Trauma-Injury Infect Crit Care 2001; 51: 452-457.

Asahi M et al. J Cerebr Blood Flow Metab 2000; 20: 1681-1689.

Bergers G. et al. Nat Cell Biol 2000; 2: 737-744.

Betsuyaku T et al. Am J Respir Cell Mol Biol 1999; 20: 1303-1309.

Betsuyaku T et al. Respiration 2000; 67: 261-267.

Carney D E et al. Circulation 1999; 100: 400-406.

Cawston T. Mol Med Today 1998; 4: 130-137.

Chadha H S et al. J Orthop. Res 1999; 17: 376-381.

Chan C C, Li Q. Br J Ophthalmol. 1998; 82: 91-96.

Colcher D et al. Q J Nucl Med 1998; 42: 225-241.

Conway J G et al. J Exp Med 1995; 182: 449-457.

Cowland J B, Borregaard N. J Leukoc Biol 1999; 66: 989-995.

Cuzner M L, Opdenakker G. J Neuroimmunol 1999; 94:1-14.

Delclaux C et al. Am J Physiol 1997; 272: L442-L451.

Dubois B et al. J Clin Invest 1999; 104: 1507-1515.

Dunican A L et al. Shock 2000; 14: 284-289.

Eichler I et al. Eur Respir J 1999; 14: 1145-1149.

Ernst P. Aliment.Pharmacol Ther 1999; 13 Suppl 1: 13-18.

Finkelstein R et al. Am J Respir Crit Care Med 1995; 152: 1666-1672.

Fisher A J et al. Am J Respir Crit Care Med 2001; 163: 259-265.

Frey B et al. Schweizerische Medizinische Wochenschrift 2000; 130: 1572-1575.

Gallin J I. Annu Rev Med 1985; 36: 263-274.

Geerts L et al. Critical Care Medicine 2001; 29: 1920-1924.

Gibbs D F et al. Am J Respir Cell Mol Biol 1999; 20: 1145-1154.

Greener, M. Neutrophils emerging role in severe asthma. Doctor's Guide. www.docguide.com. 11-17-1999.

Greener, M. Airway inflammation persists in asthma despite corticosteroids. Doctor's Guide. www.docquide.com.. 1-18-2000.

Heymans S et al. Nat Med 1999; 5: 1135-1142.

Holgate S T et al. Clin Exp Allergy 1999; 29 Suppl 2: 90-95.

Kieseier B C et al. Brain 1999; 122 (Pt 8): 1579-1587.

Koedel U, Pfister H W. Infect Dis Clin North Am 1999; 13: 549-577.

Kooman J P et al. Neth. J Med 2000; 57: 185-189.

Kuijpers T W, Weening R S, Roos D. J Immunol Meth 1999; 232: 211-229.

Leino L, Hurttia H. Clin Chem Lab Med 1999; 37: 215-222.

Leppert D et al. Clin Infect Dis 2000; 31: 80-84.

Lijnen H R et al. Thromb Haemost 1998; 79: 1171-1176.

Lindsey M et al. Circulation 2001; 103: 2181-2187.

Liu Z et al. J Exp Med 1998; 188: 475-482.

Marchevsky A M, Read R A. Aust Vet J 1999; 77: 233-237.

Mariano F et al. Kidney Int 2001; 60: 1598-1605.

Masuhara K et al. Int Orthop. 2000; 24: 92-96.

Masure S et al. Eur J Biochem 1991; 198: 391-398.

Matsukawa A, Yoshinaga M. Histol. Histopathol. 1999; 14: 511-516.

Maymon E et al. Am J Obstetr Gynecol 2000; 183: 887-894.

Mizgerd J P et al. J Exp Med 1997; 186: 1357-1364.

Nakamura T et al. Am J Med Sci 1998; 316: 355-360.

Neely A N et al., J Burn Care Rehab 2000; 21: 395-402.

Nielsen B S et al., Int J Cancer 1996; 65: 57-62.

Nurre L D, Rabalais G P, Callen J P. Pediatr. Dermatol 1999; 16: 214-216.

Paemen L et al. Eur J Biochem 1995; 234: 759-765.

Pardo A et al. Am J Respir Crit Care Med 2000; 161: 1698-1704.

Prikk K et al. J Pathol 2001; 194: 232-238.

Pruijt J F et al. Proc Natl Acad Sci USA 1999; 96: 10863-10868.

Rasmussen H S, McCann P P. i Pharmacol Ther 1997; 75: 69-75.

Ricou B et al. Am J Respir. Crit Care Med 1996; 154: 346-352.

Roberts J A. Rev Prat 1993; 43: 1063-1068.

Rotbart H A, Glode M P. Pediatrics 1985; 75: 254-259.

Sachs M K. Arch Dermatol. 1991; 127: 493-496.

Sampson A P. Clin Exp Allergy 2000; 30 Suppl 1: 22-27.

San Mateo L R et al. Infect Immun 1999; 67: 4963-4967.

Scozzafava A, Supuran C T. i J Medicin Chem 2000; 43: 3677-3687.

Sepper R et al. Chest 1995; 107: 1641-1647.

Slavkovsky, P. Inflammation. http://savba.savba.sk/logos/books/scientific/node4.html, Slovak Academy of Sciences . 6-27-1995.

Smith J R, Hart P H, Williams K A. Immunol Cell Biol 1998; 76: 497-512.

Spark J I, Scott D J A. Brit J Surg 2001; 88: 1583-1589.

Sullivan G W, Carper H T, Mandell G L. Int J Immunopharmacol. 1995; 17: 793-803.

Takei I et al. J Biomed. Mater. Res 1999; 45: 175-183.

Terui T, Ozawa M, Tagami H. Exp Dermatol 2000; 9: 1-10.

Thomas J et al. J Immunol 1997; 158: 1383-1391.

Torii K et al. Am J Respir Crit Care Med 1997; 155: 43-46.

Wang M et al. Proc Natl Acad Sci U S A 1999; 96: 6885-6889.

Wang X Y et al. J Neurosc 2000; 20: 7037-7042.

Welt F G et al. Arterioscler. Thromb Vasc. Biol 2000; 20: 2553-2558.

White J G, Clawson C C. Am J Pathol 1980; 98: 151-196.

Wilhelm S M et al. J Biol Chem 1989; 264: 17213-17221.

Wilson D M, John G R, Callen J P. J Am Acad Dermatol 1999; 40: 331-334.

Wojtowicz-Praga S et al. Invest New Drugs 1997; 15: 61-75.

Yip D et al. Investig New Drugs 1999; 17: 387-399.

Zachem C R et al. J Am Soc Nephrol. 1997; 8:1838-1844.

Zhou N et al. FEBS Lett 1997; 414: 562-566.

Ziaber J et al. Mediators Inflamm 1998; 7: 335-338.

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention.

What is claimed is:

1. A method for the prevention or treatment of a neutrophil-mediated inflammatory disorder, comprising the step of specifically neutralizing the biological activity of a neutrophil-secreted matrix metalloproteinase (MMP).

2. The method of claim 1, wherein only the biological activity of a single neutrophil-secreted MMP is neutralized.

3. The method of claim 2, wherein said neutrophil-secreted MMP is MMP-9.

4. The method of claim 3, wherein MMP-9 biological activity is neutralized with an anti-MMP-9 antibody.

5. The method of claim 4, wherein said anti-MMP-9 antibody is REGA-3G12.

6. The method of claim 4, wherein said anti-MMP-9 antibody is 3G12-scFv.

7. The method of claim 3, wherein said anti-MMP-9 antibody is coupled to an anti-inflammatory cytokine.

8. The method of claim 7, wherein said anti-inflammatory cytokine is selected from the group consisting of IL-1 receptor antagonist, IL-4, IL-6, IL-10, IL-11, IL-13, TGFβ and somatostatin.

9. The method of claim 1, wherein said neutrophil-mediated inflammatory disorder is selected from the group consisting of: septic shock, acute respiratory distress syndrome (ARDS), bacterial meningitis, acute pancreatitis, multiple organ failure (MOF), post-ischemic reperfusion, acute cellulitis, abdominal aortic aneurysm, asthma, osteomyelitis, Crohn's disease, cystic fibrosis, emphysema, septic or bacterial pyelonephritis, rheumatoid arthritis, septic arthritis, uveitis, periodontitis, psoriasis, severe burns, skin ulceration, acute lung injury, pneumonia, trauma, severe early graft dysfunction, brochioeactasis, chronic obstructive pulmonary disease (COPD), complications with hemodialysis, hypersensitivity pneumonitis, lung fibrosis, herpes stromal keratitis, vascular restenosis, glomerulonephritis and multiple sclerosis.

10. A method for the prevention or treatment of a neutrophil-mediated disease in a human or an animal, comprising administering to said human or animal a pharmaceutically effective amount of an inhibitor that neutralizes specifically the biological activity of a neutrophil-secreted matrix metalloproteinase (MMP).

11. The method of claim 10, wherein said inhibitor neutralizes only the biological activity of a single neutrophil-secreted MMP.

12. The method of claim 11, wherein said neutrophil-secreted MMP is MMP-9.

13. The method of claim 12, wherein said inhibitor is an anti-MMP-9 antibody.

14. The method of claim 13, wherein said anti-MMP-9 antibody is REGA-3G12.

15. The method of claim 13, wherein said anti-MMP-9 antibody is 3G12-scFv.

16. The method of claim 13, wherein said anti-MMP-9 neutralizing antibody is coupled to an anti-inflammatory cytokine.

17. The method of claim 16, wherein said anti-inflammatory cytokine is selected from the group consisting of IL-1 receptor antagonist, IL-4, IL-6, IL-10, IL-11, IL-13, TGFβ and somatostatin.

18. The method of claim 11, further comprising administering to said human or animal an anti-inflammatory cytokine.

19. The method of claim 11, wherein said neutrophil-mediated disease is selected from the group consisting of: septic shock, acute respiratory distress syndrome (ARDS), bacterial meningitis, acute pancreatitis, multiple organ failure (MOF), post-ischemic reperfusion, acute cellulitis, abdominal aortic aneurysm, asthma, osteomyelitis, Crohn's disease, cystic fibrosis, emphysema, septic or bacterial pyelonephritis, rheumatoid arthritis, septic arthritis, uveitis, periodontitis, psoriasis, severe burns, skin ulceration, acute lung injury, pneumonia, trauma, severe early graft dysfunction, brochioeactasis, chronic obstructive pulmonary disease (COPD), complications with hemodialysis, hypersensitivity pneumonitis, lung fibrosis, herpes stromal keratitis, vascular restenosis, glomerulonephritis, hypersensitivity, cardiac rupture arising as a complication of myocardial infarction, multiple sclerosis, stroke and cerebral ischemia, and traumatic brain injury.
